(19) United States
(12) Patent Application Publication
(10) Pub. No.: US 2026/0348141 A1
(43) Pub. Date: Nov. 5, 2026

(54) DEVICE FOR ADDING FLUID MEDIUM TO SHOWER SYSTEM, AND SHOWER SYSTEM

(71) Applicant: Magid Abdalla Suliman, Blackrock (IE)

(72) Inventor: Magid Abdalla Suliman, Blackrock (IE)

(21) Appl. No.: 19/739,187

(22) Filed: Jul. 13, 2026

Related U.S. Application Data
(63) Continuation-in-part of application No. 18/905,316, filed on Oct. 3, 2024, now Pat. No. 12,709,882.
Application No. 18/905,316 is a continuation-in-part of application No. 17/250,803, filed on Oct. 24, 2019, now Pat. No. 12,134,883.

(30) Foreign Application Priority Data
Nov. 1, 2018 (CN) 201821794448.5

(51) Int. Cl. E03C 1/046; A47K 3/28; A61L 2/10; A61L 103/00; B08B 9/032; E03C 1/02; E03C 1/04; F16K 17/04; F16K 17/164; G05D 16/00; G05D 16/10; G05D 16/20

(52) U.S. Cl. CPC E03C 1/046; A47K 3/281; A61L 2/10; B08B 9/0325; E03C 1/025; E03C 1/0408; F16K 17/04; F16K 17/164; G05D 16/024; G05D 16/10; G05D 16/2066; A61L 2103/23; B08B 2209/032; E03C 2201/40

(57) ABSTRACT

A device for adding a fluid medium to a shower system includes a water pipe assembly, a fluid medium pipe assembly, and a pressure regulating assembly. The fluid medium pipe assembly includes a suction pipe and a switch valve, the suction pipe is provided with a fluid medium inlet and a fluid medium outlet that is connected with the water pipe assembly. The pressure regulating assembly includes a pressure sensor and a pressure relief valve. The pressure sensor is configured to collect a water pressure signal in the water pipe assembly. The pressure relief valve is configured to: actively open when the water pressure in the water pipe assembly exceeds a first set threshold; and when the water pressure signal collected by the pressure sensor exceeds a second set threshold, control a first water pump to stop working. The first set threshold is greater than the second set threshold.

### CROSS-REFERENCE TO RELATED APPLICATION(S)

This application is a continuation-in-part of U.S. patent application Ser. No. 18/905,316, filed on Oct. 3, 2024, which is a continuation-in-part of U.S. patent application Ser. No. 17/250,803, filed Mar. 4, 2021, a Section 371 National Stage Application of International Application No. PCT/CN2019/112922, filed Oct. 24, 2019 and published as WO 2020/088335 A1 on May 7, 2020, which further claims priority to Chinese Patent Application Ser. No. CN201821794448.5, filed on Nov. 1, 2018, the entire disclosure of each of which is incorporated herein by reference in entirety.

### TECHNICAL FIELD

The present application relates to the technical field of shower system, and more particularly, to a device for adding a fluid medium to a shower system, and a shower system.

### BACKGROUND

The existing shower system generally includes a water heater, a shower and a water mixing valve. An inlet of the water mixing valve is connected with an outlet of the water heater, the other inlet of the water mixing valve is connected with a cold water pipe, and an outlet of the water mixing valve is connected with the shower. Hot water in the water heater is mixed with cold water in the cold water pipe in the water mixing valve, and then is transported to the shower. Modern people habitually use water from the shower system for spraying or washing, and apply shower gel or soap on the skin during the shower to help personal hygiene. For a long time, people take shower gel through directly squeezing the bottle containing the shower gel by hand, and then apply it to the skin so that it can be mixed with water from the shower system or can be used for washing, which is tedious and troublesome.

### SUMMARY

The present application aims to solve at least one of the technical problems existing in the prior art. To this end, the present application proposes a device for adding a fluid medium to a shower system. The device for adding the fluid medium to the shower system is simple in structure, and enables a user to control the supply of the fluid medium easily and conveniently while showering.

In a first aspect, embodiments of the present application provide a device for adding a fluid medium to a shower system. The device includes:
a water pipe assembly, including a water pipe; wherein the water pipe has a water inlet and a water outlet, the water inlet is configured to be connected with a water source connector of the shower system for introducing water, and the water outlet is configured to be connected with a showerhead of the shower system;
a fluid medium pipe assembly, including a suction pipe; wherein the suction pipe has a fluid medium inlet and a fluid medium outlet, and the fluid medium outlet is connected with the water pipe assembly;
a reservoir for storing the fluid medium; wherein the suction pipe extends into the reservoir;
a valve body assembly, including a first water pump and a three-way valve pipe; wherein the water inlet is in communication with a water inlet pipe of the three-way valve pipe through the first water pump,
a water return pipe of the three-way valve pipe is in communication with the fluid medium outlet, and a water mixing pipe of the three-way valve pipe is in communication with the water outlet;
a pressure regulating assembly, including a pressure sensor and a pressure relief valve; wherein the pressure sensor is configured to collect a water pressure signal of the water pipe assembly, and the pressure relief valve is disposed at a water inlet side and is configured to actively open in a case where the water pressure signal in the water pipe assembly exceeds a first set threshold, for discharging at least part of fluid in the water pipe assembly; and
a control system, including a main control module and a power supply module; wherein an output end of the power supply module is respectively connected to a power supply end of the main control module, a power supply end of the pressure sensor, and a power supply end of the first water pump; an output end of the pressure sensor is connected to an input end of the main control module, and an output end of the main control module is connected to an input end of the first water pump; wherein the control system is configured to: in a case where the water pressure signal collected by the pressure sensor exceeds a second set threshold, control the first water pump to stop working; wherein the first set threshold is greater than the second set threshold.

In a second aspect, embodiments of the present application provide a device for adding a fluid medium to a shower system. The device includes:
a water pipe assembly, including a water pipe; wherein the water pipe has a water inlet and a water outlet, the water inlet is configured to be connected with a water source connector of the shower system for introducing water, and the water outlet is configured to be connected with a showerhead of the shower system;
a reservoir for storing the fluid medium; wherein the reservoir includes a storage chamber and a cleaning chamber that are independently arranged; the storage chamber is configured to accommodate a care medium for body washing of a user, and the cleaning chamber is configured to accommodate a cleaning agent for cleaning pipelines of the device;
a fluid medium pipe assembly, including a suction pipe and a switch valve; wherein the suction pipe has a fluid medium inlet and a fluid medium outlet, and the fluid medium outlet is connected with the water pipe assembly; wherein the suction pipe includes a communication portion, a care medium suction portion, and a cleaning agent suction portion; a first end of the care medium suction portion extends into the storage chamber, and a second end of the care medium suction portion is connected with a liquid inlet end of the communication portion; a first end of the cleaning agent suction portion extends into the cleaning chamber, and a second end of the cleaning agent suction portion is connected with the liquid inlet end of the communication portion; the first end of the care medium suction portion and the first end of the cleaning agent suction portion respectively constitute the fluid medium inlet, and a liquid outlet end of the communication portion constitutes the fluid medium outlet; the switch valve is configured to allow the communication portion to be selectively in communication with the care medium suction portion or the cleaning agent suction portion;
a valve body assembly, including a first water pump and a three-way valve pipe; wherein the water inlet is in communication with a water inlet pipe of the three-way valve pipe through the first water pump, a water return pipe of the three-way valve pipe is in communication with the fluid medium outlet, and a water mixing pipe of the three-way valve pipe is in communication with the water outlet; and
a control system, including a main control module and a power supply module; wherein an output end of the power supply module is respectively connected to a power supply end of the main control module, a power supply end of the switch valve, and a power supply end of the first water pump, and an output end of the main control module is connected to an input end of the switch valve;
wherein the control system is configured to: upon receiving a first signal indicating that the user has finished showering or a second signal indicating that a standing time after the user finishes showering reaches a first preset delay threshold, control the switch valve to allow the communication portion to be in communication with the cleaning agent suction portion, and control the first water pump to work, for causing the cleaning agent in the cleaning chamber to flow into the water pipe assembly to clean at least part of pipelines of the water pipe assembly.

In a third aspect, embodiments of the present application provide a device for adding a fluid medium to a shower system. The device includes: a box body and a liquid storage assembly; wherein the box body and the liquid storage assembly are detachably connected, and the box body is provided with:
a water pipe assembly, including a water pipe; wherein the water pipe has a water inlet and a water outlet; the water inlet is configured to be connected with a water source connector of the shower system for introducing water, and the water outlet is configured to be connected with a showerhead of the shower system;
a fluid medium pipe assembly, including a suction pipe and a switch valve; wherein the suction pipe has a fluid medium outlet and a plurality of fluid medium inlets, and the plurality of fluid medium inlets are mutually independent; the fluid medium outlet is connected with the water pipe assembly, a communication portion is provided inside the suction pipe, and the plurality of fluid medium inlets are respectively in communication with the fluid medium outlet via the communication portion; the switch valve is configured to switch a flow path of the communication portion, for causing the communication portion to selectively connect any one or more of the the plurality of fluid medium inlets with the fluid medium outlet; and
a valve body assembly, including a first water pump and a three-way valve pipe; wherein the water inlet is in communication with a water inlet pipe of the three-way valve pipe through the first water pump, a water return pipe of the three-way valve pipe is in communication with the fluid medium outlet, and a water mixing pipe of the three-way valve pipe is in communication with the water outlet;
wherein the liquid storage assembly includes a plurality of consumable cartridges that are mutually independent, and each consumable cartridge has a chamber for placing a fluid medium; wherein the box body is provided with a plurality of connection positions for detachably connecting with the plurality of consumable cartridges, and the plurality of consumable cartridges are capable of being detachably connected to the plurality of connection positions in a one-to-one correspondence, enabling each fluid medium inlet of the suction pipe extends into the chamber of a corresponding consumable cartridge.

To this end, when the device for adding a fluid medium to a shower system according to the embodiments of the present application is used by a user, the fluid medium is controlled to be sucked from the fluid medium inlet of the suction pipe and flow into the water return pipe of the three-way valve pipe. The fluid medium is mixed with the water flowing into the water inlet pipe of the three-way valve pipe in the water mixing pipe of the three-way valve pipe, and then flows into the shower system via the water outlet to supply the fluid medium for the user. In the above operations, the fluid medium can be brought into contact with the skin of the user without directly squeezing the bottle containing the fluid medium by hand.

The additional aspects and advantages of the present application will be partly given in the following description, and some of the additional aspects and advantages will become obvious from the following description, or be understood through the practice of the present application.

### BRIEF DESCRIPTION OF THE DRAWINGS

In order to illustrate technical solutions provided in embodiments of the present application or in the prior art more clearly, the disclosure will be further elaborated with the drawings for illustrating the embodiments or the prior art. It is apparent that the drawings in the following descriptions only show some embodiments of the present application. Other drawings may further be obtained by those of ordinary skill in the art according to these drawings without creative work.

The present application is a continuation-in-part of a prior parent application; wherein FIGS. 1-6 correspond to the first embodiment disclosed in the parent application; FIGS. 7-11 correspond to the second embodiment disclosed in the parent application; FIGS. 12-15 correspond to the third, fourth, and fifth embodiments newly added in the present continuation-in-part application, which are reservoir variant structures and matching adjusted control system connection diagrams. Each newly added embodiment mainly differs from the first and second embodiments of the parent application in the improved reservoir structure and correspondingly adapted control system logic; the overall pipeline and reversing valve basic structure follow the design concepts of the first and second embodiments of the parent application.

FIG. 1 is a perspective view of a device for adding a fluid medium to a shower system according to a first embodiment of the present application;

FIG. 2 is a perspective view of the first embodiment shown in FIG. 1 from another perspective;

FIG. 3 is a perspective view illustrating the connection of a pipe assembly and a valve body assembly of the first embodiment shown in FIG. 1;

FIG. 4 is a front view of a reservoir according to a first embodiment of the present application;

FIG. 5 is a schematic diagram of a reversing valve assembly of the first embodiment shown in FIG. 1;

FIG. 6 is a schematic diagram of a control system of the first embodiment shown in FIG. 1;

FIG. 7 is a perspective view of a device for adding a fluid medium to a shower system according to a second embodiment of the present application;

FIG. 8 is a perspective view of the second embodiment shown in FIG. 7 from another perspective;

FIG. 9 is a perspective view illustrating the connection of a pipe assembly and a valve body assembly of the second embodiment shown in FIG. 7;

FIG. 10 is a front view of a bottom plate and a fixing plate of the second embodiment shown in FIG. 7; and

FIG. 11 is a perspective view illustrating the assembly of a bottom plate, a pipe assembly and a valve body assembly of the second embodiment shown in FIG. 7;

FIG. 12 is a schematic diagram of a control system of a device for adding a fluid medium to a shower system according to a third embodiment of the present application;

FIG. 13 is a schematic diagram of a control system of a device for adding a fluid medium to a shower system according to a fourth embodiment of the present application;

FIG. 14 is a front view of a reservoir according to the fourth embodiment of the present application;

FIG. 15 is a front view of a liquid storage assembly of a device for adding a fluid medium to a shower system according to a fifth embodiment of the present application.

### LIST OF REFERENCE SYMBOLS

device 100 for adding a fluid medium to a shower system,
water pipe assembly 10, water pipe 11, water inlet 11a, water outlet 11b, bypass pipe 12,
water inlet end 12a, water outlet end 12b, flow regulator 13, second activation element 13a, second rotary knob 13b, first one-way valve 14,
fluid medium pipe assembly 20, suction pipe 21, fluid medium inlet 21a, fluid medium outlet 21b, first suction portion 21c, second suction portion 21d, communication portion 21e, cleaning agent suction portion 21f, venturi switch 22, first activation element 22a, first rotary knob 22b, switch valve 23,
valve body assembly 30, three-way valve pipe 31, water inlet pipe 31a, water return pipe 31b, water mixing pipe 31c, second one-way valve 32, first water pump 33,
pressure regulating assembly 34, pressure sensor 34a, ultraviolet sterilization module 35;
box body 40, through hole 41a
liquid storage assembly 500, first reservoir 50, first protrusion 51, second reservoir 52, first chamber 52a, second chamber 52b, second protrusion 52, second protrusion 53, cleaning chamber 54, consumable cartridge 55;
fixing plate 60, first groove portion 61,
bottom plate 70, second groove portion 71, third groove portion 72,
connecting plate 80′,
control system 80, controller 81, battery 82, radio frequency receiver 83,
reversing valve assembly 90, first valve port 91, second valve port 92, third valve port 93, fourth valve port 94, second water pump 95, first on-off valve 96, second on-off valve 97.

The implementations of the purposes, functional characteristics, and advantages of the present application will be further described in conjunction with the embodiments and with reference to the drawings.

### DETAILED DESCRIPTION

The embodiments of the present application will be described in detail below. Examples of the embodiments are shown in the drawings in which the same or similar reference symbols represent the same or similar elements or elements with the same or similar functions. It is to be understood that the embodiments described below with reference to the drawings are exemplary, and are merely intended to illustrate the present application, but are not be intended to limit the present application.

The following description provides many different embodiments or examples for implementing different structures of the present application. In order to simplify the disclosure of the present application, the components and arrangements of the specific examples are described below. Of course, they are only exemplary and are not intended to limit the present application. In addition, reference symbols and/or letters may be repeated in the various examples of the present application. This repetition is for the purpose of simplicity and clarity and does not indicate a relationship between the various embodiments and/or arrangements discussed. Furthermore, various specific processes and materials are provided in the present application, but it is known for those of ordinary skill in the art that other processes and/or other materials may be used.

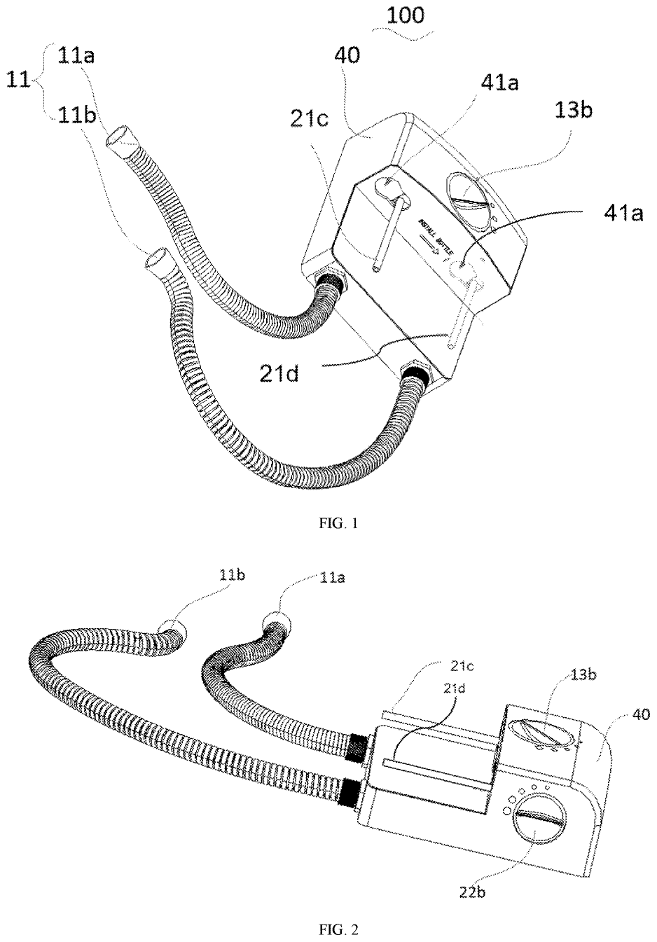

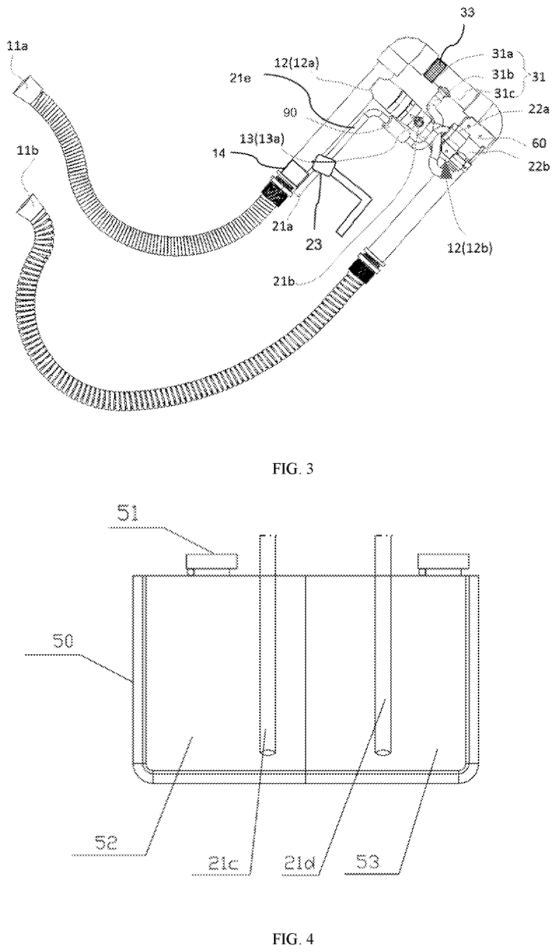

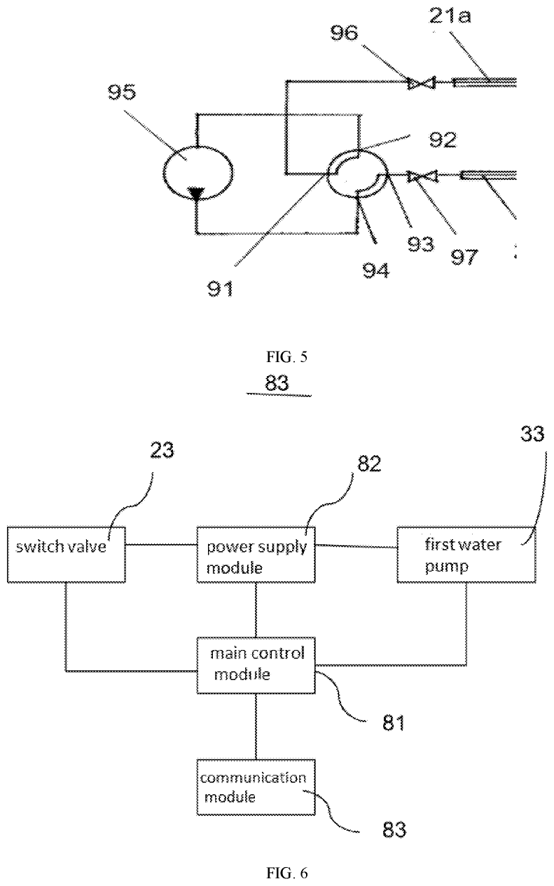

A device 100 for adding a fluid medium to a shower system according to a first embodiment of the present application will be described below with reference to FIGS. 1-6.

The device 100 for adding a fluid medium to a shower system according to the first embodiment of the present application includes a water pipe assembly 10, a first reservoir 50 for storing the fluid medium, a fluid medium pipe assembly 20, and a valve body assembly 30. The water pipe assembly 10 includes a water pipe 11 provided with a water inlet 11a and a water outlet 11b, and the water inlet 11a and the water outlet 11b are configured to be connected with the shower system. The second reservoir 52 includes a first chamber 52a and a second chamber 52b which are separate from each other in a left-right direction, the first chamber 52a is configured to accommodate a first fluid medium, and the second chamber 52b is configured to accommodate a second fluid medium different from the first fluid medium. The fluid medium pipe assembly 20 includes a suction pipe 21 and a switch valve 23, the suction pipe 21 is provided with a fluid medium inlet 21a and a fluid medium outlet 21b, and the fluid medium outlet 21b is connected with the water pipe assembly 10. The suction pipe 21 includes a communication portion 21e, a first suction portion 21c, and a second suction portion 21d. A first end of the first suction portion 21c extends into the first chamber 52a, and a second end of the first suction portion 21c is connected with a liquid inlet end of the communication portion 21e. A first end of the second suction portion 21d extends into the second chamber 52b, and a second end of the second suction portion 21d is connected with the liquid inlet end of the communication portion 21e. The first end of the first suction portion 21c and the second end of the second suction portion 21d form the fluid medium inlet 21a, and a liquid outlet end of the communication portion 21e forms the fluid medium outlet 21b. The switch valve 23 is configured to allow the communication portion 21e to be selectively in communication with the first suction portion 21c or the second suction portion 21d. The valve body assembly 30 includes a first water pump 33 and a three-way valve pipe 31. The water inlet 11a is in communication with a water inlet pipe 31a of the three-way valve pipe 31 through the first water pump 33, a water return pipe 31b of the three-way valve pipe 31 is in communication with the fluid medium outlet 21b, and a water mixing pipe 31c of the three-way valve pipe 31 is in communication with the water outlet 11b. The water pipe assembly further includes a bypass pipe 12 and a flow regulator 13. A water inlet end 12a of the bypass pipe 12 is in communication with the water inlet 11a and the water inlet pipe 31a, and a water outlet end 12b of the bypass pipe 12 is in communication with the water outlet 11b and the water mixing pipe 31c. The flow regulator 13 is provided between the water inlet end 12a of the bypass pipe 12 and the water outlet end 12b of the bypass pipe 12.

Thereby, when the device 100 for adding a fluid medium to a shower system according to the first embodiment of the present application is used by a user, a negative pressure is generated in the water inlet pipe 31a of the three-way valve pipe 31 of the device of the present application, so as to control a fluid medium to be sucked from a fluid medium inlet 21a of the suction pipe 21 and flow into a water return pipe 31b of the three-way valve pipe 31. The fluid medium is mixed with the water flowing into the water inlet pipe 31a of the three-way valve pipe 31 in the water mixing pipe 31c of the three-way valve pipe 31, and then flows into the shower system via the water outlet 11b to supply the fluid medium for the user. The design of the bypass pipe 12 can enhance the injection effect of the three-way valve pipe 31. The flow regulator 13 is provided between the water inlet end 12a of the bypass pipe 12 and the water outlet end 12b of the bypass pipe 12. The design of the flow regulator 13 can control the strength of the injection effect of the three-way valve pipe 31. The first water pump 33 can provide power for water. The first chamber 52a and the second chamber 52b accommodate different fluid mediums. The user can control whether the first fluid medium accommodated in the first chamber 52a flows into the suction pipe 21 or the second fluid medium accommodated in the second chamber 52b flows into the suction pipe 21 by controlling the switch valve 23, to supply the user. The device 100 of the embodiments of the present application can stably and reliably provide the fluid medium to the user.

In some specific examples of the present application, the water pipe assembly 10 includes a first one-way valve 14 for preventing a back flow of the water, and the first one-way valve 14 is provided between the water inlet 11a and the water inlet end 12a of the bypass pipe 12. The device 100 of the embodiment of the present application can prevent water from flowing back.

In some specific examples of the present application, a front face or a side face of the second reservoir 52 is provided with an opening configured to allow a gel or tablet to be dispensed into the first chamber 52a and/or the second chamber 52b. The second reservoir 52 further includes a door configured to open or close the second reservoir 52. The door allows the corresponding first chamber 52a or second chamber 52b to form a sealed environment when closing the opening of the second reservoir 52. When the user uses the device 100 of the present embodiment, a bath gel, a shampoo gel, a bath tablet (such as a bath effervescent tablet), or a shampoo tablet (such as a shampoo effervescent tablet) can be dispensed through the opening of the second reservoir 52, and the opening of the second reservoir 52 can be closed by the door. It will be appreciated that when the user dispenses a bath tablet (such as a bath effervescent tablet) or a shampoo tablet (such as a shampoo effervescent tablet) through the opening of the second reservoir 52, the user may also pour water inward through the opening of the second reservoir 52 to allow the tablet to be decomposed in the water to form a fluid medium.

In some specific examples of the present application, the device 100 further includes a reversing valve assembly 90 configured to control a flow direction. The reversing valve assembly 90 is disposed between the fluid medium inlet 21a and the fluid medium outlet 21b. The reversing valve assembly 90 is configured to control water to flow from the fluid medium outlet 21b to the fluid medium inlet, and the reversing valve assembly 90 is further configured to control the fluid medium to flow from the fluid medium inlet to the fluid medium outlet 21b. It will be appreciated that when the user dispenses a bath tablet (such as a bath effervescent tablet) or a shampoo tablet (such as a shampoo effervescent tablet) through the opening of the second reservoir 52, the reversing valve assembly 90 may control the water to flow from the fluid medium outlet 21b to the fluid medium inlet, to allow the tablet in the f second reservoir 52 to be decomposed in the water to form the fluid medium.

In some specific examples of the present application, the bottom of the box body 40 is provided with a through hole 41a, and the second reservoir 52 is provided with a second protrusion 53 that is plug-fitted with the through hole 41a. The installation and removal of the box body 40 and the second reservoir 52 may be achieved by pulling and pushing.

In some specific examples of the present application, the reversing valve assembly 90 includes a first valve port 91, a second valve port 92, a third valve port 93, a fourth valve port 94, a second water pump 95, a first on-off valve 96, and a second on-off valve 97. The first valve port 91 is connected with an inlet of the second water pump 95, the third valve port 93 is connected with an outlet of the second water pump 95, the second valve port 92 is connected with the first on-off valve 96, the fourth valve port 94 is connected with the second on-off valve 97, the first on-off valve 96 is connected with the fluid medium inlet 21a, and the second on-off valve 97 is connected with the fluid medium outlet 21b. When the user dispenses a tablet into the first chamber 52a, the reversing valve assembly 90 is configured to allow the third valve port 93 to be in communication with the second valve port 92 and to allow the first valve port 91 to be in communication with the fourth valve port 94, and the second water pump 95 is configured to allow water to flow from the fluid medium outlet 21b to the fluid medium inlet to flow into the second reservoir 52; the reversing valve assembly 90 is configured to allow the third valve port 93 to be in communication with the first valve port 91 and allow the second valve port 92 to be in communication with the fourth valve port 94, and the second water pump 95 is configured to allow the fluid medium water in the second reservoir 52 to flow from the fluid medium inlet to the fluid medium outlet 21b.

In some specific examples of the present application, the device 100 further includes a control system 80 including a controller 81 and a battery 82. An output end of the battery 82 is connected with a power supply end of the controller 81, a power supply end of the switch valve 23, and a power supply end of the first water pump 33. An output end of the controller 81 is connected with an input end of the first water pump 33 and an input end of the switch valve 23. When the user uses the device 100 of the present embodiment, the controller 81 can control the operation of the first water pump 33 to control the flow rate of water or the working state of the device 100. The user can also control the operation of the switch valve 23 through the controller 81 to control whether the first fluid medium accommodated in the first chamber 52a flows into the suction pipe 21 or the second fluid medium accommodated in the second chamber 52b flows into the suction pipe 21, to supply the user.

In some specific examples of the present invention, the control system 80 further includes a radio frequency receiver 83. The output end of the battery 82 is connected with a power supply end of the radio frequency receiver 83, and the switch valve 23 is connected with the radio frequency receiver 83 through the controller 81. The radio frequency receiver83 is configured to acquire a first command for controlling the communication portion 21e to be in communication with the first suction portion 21c or the second suction portion 21d, and the controller 81 controls the operation of the switch valve 23 according to the first command. In some specific examples of the present application, the control system 80 further includes a radio frequency receiver 83. The output end of the battery 82 is connected with a power supply end of the radio frequency receiver 83, and the first water pump 33 is connected with the radio frequency receiver 83 through the controller 81. The radio frequency receiver 83 is configured to acquire a second command for controlling the first water pump 33, and the controller 81 controls the operation of the first water pump 33 according to the second command. The user may transmit the first command or the second command of a mobile app or a remote controller to the controller 81 through the radio frequency receiver 83. This design improves the user experience.

In some specific examples of the present application, an outer surface of at least one of the water pipe 11, the suction pipe 21, the valve body assembly 30 or the bypass pipe 12 is at least partially covered with an antimicrobial material.

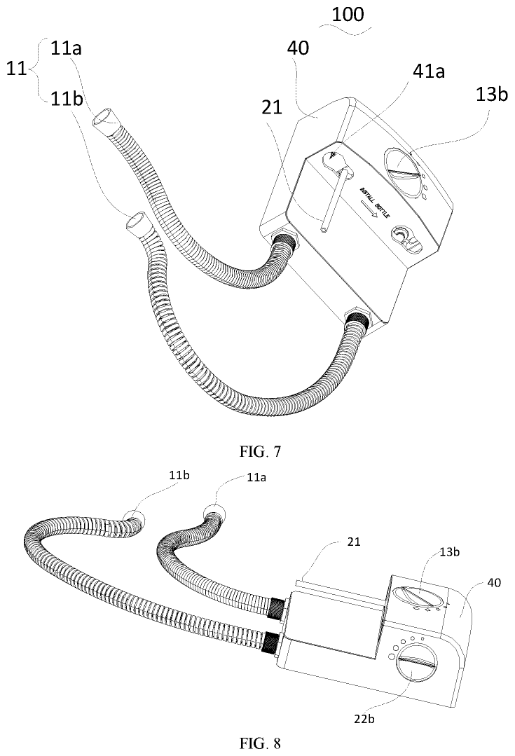

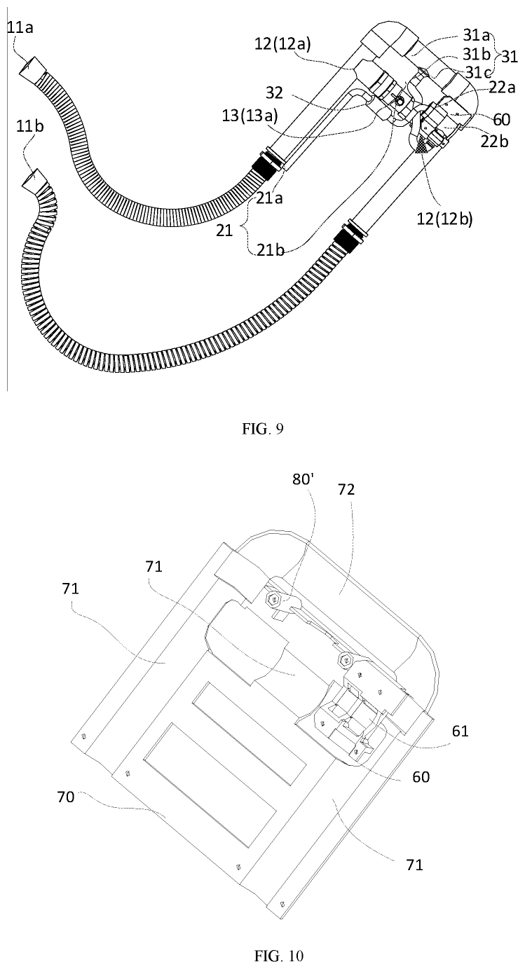

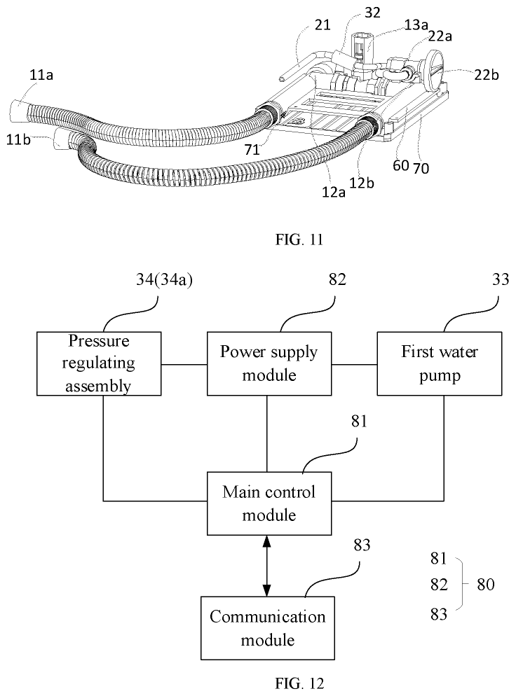

A device 100 for adding a fluid medium to a shower system according to a second embodiment of the present application will be described below with reference to FIGS. 7 to 11.

The device 100 for adding a fluid medium to a shower system according to the second embodiment of the present application includes a water pipe assembly 10, a fluid medium pipe assembly 20, and a valve body assembly 30. The water pipe assembly 10 includes a water pipe 11 provided with a water inlet 11a and a water outlet 11b, and the water inlet 11a and the water outlet 11b are configured to be connected with the shower system. The fluid medium pipe assembly 20 includes a suction pipe 21, the suction pipe 21 is provided with a fluid medium inlet 21a and a fluid medium outlet 21b, and the fluid medium outlet 21b is connected with the water pipe assembly 10. The valve body assembly 30 includes a second one-way valve 32 for preventing a back flow of the fluid medium and a three-way valve pipe 31. The second one-way valve 32 is provided between the fluid medium inlet 21a and the fluid medium outlet 21b. The three-way valve pipe 31 includes a three-way valve pipe. A water inlet pipe 31a of the three-way valve pipe is in communication with the water inlet 11a, a water return pipe 31b of the three-way valve pipe is in communication with the fluid medium outlet 21b, and a water mixing pipe 31c of the three-way valve pipe is in communication with the water outlet 11b.

Thereby, when the device 100 for adding a fluid medium to a shower system according to the second embodiment of the present application is used by a user, a negative pressure is generated in the water inlet pipe 31a of the three-way valve pipe 31 of the device of the present application, so as to control a fluid medium to be sucked from the fluid medium inlet 21a of the suction pipe 21 and flow into the water return pipe 31b of the three-way valve pipe 31. The fluid medium is mixed with the water flowing into the water inlet pipe 31a of the three-way valve pipe 31 in the water mixing pipe 31c of the three-way valve pipe 31, and then flows into the shower system via the water outlet 11b to supply the fluid medium for the user. In the above operations, the fluid medium can be brought into contact the skin of the user without directly squeezing the bottle containing the fluid medium by hand.

In addition, the device 100 for adding a fluid medium to a shower system according to the embodiment of the present application can achieve at least some of the following effects.

Reliability is good. The device according to the embodiment of the present application is provided with a second one-way valve 32 between the fluid medium inlet 21a and the fluid medium outlet 21b. Therefore, such device of the present application can prevent the fluid medium from flowing back and prevent water from flowing out of the fluid medium inlet 21a and in turn contaminating the fluid medium.

The versatility is good. The device according to the embodiment of the present application can be directly installed in the existing shower system, which can easily and quickly solve the cumbersome cleaning work of the user.

It is understood for those skilled in the art that the fluid medium used by people when showering includes perfume, soapy water, shampoo, shower gel or other skin care products in form of fluid.

The device according to the embodiment of the present application can be used in any shower system, such as a shower system with a shower room, a shower at a floor level, a large-capacity shower, or the like. The device according to the embodiment of the present application can also be integrated into a bathtub.

In some specific examples of the present application, the three-way valve pipe includes a venturi nozzle. The water inlet pipe 31a, the water return pipe 31b and the water mixing pipe 31c meet at a center (not shown), and the venturi nozzle is provided between the water inlet pipe 31a and the center. The venturi nozzle has a continuously reduced cross-sectional area of a flow channel, which can form a low pressure in the water inlet pipe 31a, thereby sucking the fluid medium transported and mixing it with water in the water mixing pipe 31c. The device according to the embodiments of the present application has a strong self-suction capability and can reliably suck the fluid medium into the water pipe 11.

In some specific examples of the present application, the three-way valve pipe is a venturi three-way valve pipe. The venturi three-way valve pipe has a venturi nozzle that continuously reduces a cross-sectional area of a flow channel and a venturi diffusion chamber that continuously increases the cross-sectional area of the flow channel (the venturi diffusion chamber is located between the center and the water outlet 11b). Therefore, the device according to the embodiments of the present application has a better self-suction capability. In addition, the venturi diffusion chamber is relatively insensitive to the impurities, and thus is especially suitable as a discharge pipe for the mixed liquid after the fluid medium is mixed with water.

In some specific examples of the present application, the fluid medium pipe assembly 20 includes a venturi switch 22, and the venturi switch 22 is provided between the fluid medium inlet 21a and the water return pipe 31b. The venturi switch 22 may be used to control the opening and closing of the valve body assembly 30, thereby meeting the needs of a user to start or stop using the mixed liquid after the fluid medium is mixed with water.

In some specific examples of the present application, the device includes a box body 40. The fluid medium pipe assembly 20 and the valve body assembly 30 are disposed in the box body 40. The box body 40 is designed to protect the fluid medium pipe assembly and the valve body assembly 30 from being damaged by external forces and also for aesthetics.

In some specific examples of the present application, the device includes the box body 40 and a fixing plate 60. The fixing plate 60 has a first groove portion 61. The venturi switch 22 includes a first activation element 22a, and the first activation element 22a is disposed in the first groove portion 61 and faces a left side face or a right side face of the box body 40. When the device according to the embodiments of the present application is hung on a wall with a height of about 1.5 meters from the ground, the activation element of the venturi switch 22 is arranged on the side face of the box body 40, conforming to human mechanics.

Those skilled in the art can understand that when the device according to the embodiments of the present application is hung on a wall with a height of about 0.5 meters from the ground, the activation element of the venturi switch 22 is arranged on the top face of the box body 40, conforming to human mechanics

In some specific examples of the present application, the venturi switch 22 further includes a first switching element (not shown) and a first rotary knob 22b. The first rotary knob 22b is connected with the first activation element 22a, and the first rotary knob 22b protrudes from the box body 40 to facilitate the user operation.

In some specific examples of the present application, the water pipe assembly 10 includes a bypass pipe 12. A water inlet end 12a of the bypass pipe 12 is in communication with the water inlet 11a and the water inlet pipe 31a respectively, and a water outlet end 12b of the bypass pipe 12 is in communication with the water outlet 11b and the water mixing pipe 31c respectively. The design of the bypass pipe 12 can enhance the injection effect of the three-way valve pipe 31. The device of the embodiments of the present application can stably and reliably provide the fluid medium to the user.

Before the inventors designed a water pipe assembly 10 provided by the embodiments of the present application to include a bypass pipe 12, the device provided the embodiments of the present application is difficult to reliably supply the fluid medium to the user when the three-way valve pipe 31 is damaged or the user's home water pressure is insufficient. In order to solve the above-mentioned problem, the inventors herein have designed a device 100 for adding a fluid medium to a shower system including a bypass pipe 12 in an embodiment of the present application.

In some specific examples of the present application, the water pipe assembly 10 includes a flow regulator 13, and the flow regulator 13 is provided between the water inlet end 12a of the bypass pipe 12 and the water outlet end 12b of the bypass pipe 12. The flow regulator 13 is designed to control the strength of the injection effect of the three-way valve pipe 31, so that the device of the embodiment of the present application can stably and reliably supply the fluid medium for the user.

In some specific examples of the present application, the flow regulator 13 further includes a second activation element 13a, a second switching element (not shown), and a second rotary knob 13b. The second activation element 13a is connected with the second rotary knob 13b, and the second rotary knob 13b protrudes from the box body 40 to facilitate the user operation.

In some specific examples of the present application, the second rotary knob 13b is provided on the front of the box body 40, conforming to human mechanics.

In some specific examples of the present application, the bottom of the box body 40 is provided with a first reservoir 50 for storing the fluid medium. The suction pipe 21 is extends into the first reservoir 50. When the user uses the device according to the embodiment of the present application, the negative pressure is generated in the water inlet pipe 31a of the three-way valve pipe 31, thereby sucking the fluid medium in the first reservoir 50 via the fluid medium inlet 21a of the suction pipe 21.

In some specific examples of the present application, the box body 40 is detachably connected with the first reservoir 50. This design is convenient for the user to place the fluid medium in the first reservoir 50.

In some specific examples of the present application, the bottom of the box body 40 is provided with a through hole 41a, and the first reservoir 50 is provided with a first protrusion 51 that is plug-fitted with the through hole 41a. The installation and removal of the box body 40 and the first reservoir 50 may be achieved by pulling and pushing.

In some specific examples of the present application, the suction pipe 21 is a flexible pipe. This design facilitates the installation and removal of the box body 40 and the first reservoir 50. The first reservoir 50 and the second reservoir 52 may be the same component or different components.

In some specific examples of the present application, the device includes a bottom plate 70. The bottom plate 70 is provided with a second groove portion 71 and a third groove portion 72, the water pipe 11 is disposed in the second groove portion 71, and the three-way valve pipe 31 is disposed in the third groove portion 72. The design of the second groove portion 71 and the third groove portion 72 on the bottom plate 70 facilitates the installation and fixing of the water pipe 11 and the three-way valve pipe 31, and the device according to the embodiments of the present application will not scatter during the transportation and use.

In some specific examples of the present application, the box body 40 includes a box cover and a box bottom. The box cover and the box bottom are connected by a screw and/or a buckle. The bottom face of the box bottom has a through hole for hanging on the wall. The bottom plate 70 is fixedly connected with the box bottom. The fixing plate 60 is provided on the bottom plate 70.

In some specific examples of the present application, the device includes a connecting plate 80′ fixedly connected with the bottom plate 70. The connecting plate 80′ is provided with a screw hole for connection with a screw of the box cover.

In some specific examples of the present application, the fluid medium includes one or more fragrance medium. Those skilled in the art can understand that the fluid medium used by people when showering includes perfume, soapy water, shampoo, shower gel or other skin care products in form of fluid.

A device 100 for adding a fluid medium to a shower system according to a third embodiment of the present application will be described below with reference to FIG. 12 and FIGS. 1-11.

The device 100 for adding a fluid medium to a shower system according to the third embodiment of the present application includes: a water pipe assembly 10. The water pipe assembly 10 includes a water pipe 11, and the water pipe 11 has a water inlet 11a and a water outlet 11b, where the water inlet 11a is configured to be connected with a water source connector (not shown) of the shower system for introducing water, and the water outlet 11b is configured to be connected with a showerhead (not shown) of the shower system. The device 100 further includes a fluid medium pipe assembly 20. The fluid medium pipe assembly 20 includes a suction pipe 21, the suction pipe 21 has a fluid medium inlet 21a and a fluid medium outlet 21b, and the fluid medium outlet 21b is connected with the water pipe assembly 10. The device 100 further includes a reservoir 50, the reservoir 50 is for storing the fluid medium, and the suction pipe 21 extends into the reservoir 50. The device 100 further includes a valve body assembly 30, and the valve body assembly 30 includes a first water pump 33 and a three-way valve pipe 31. The water inlet 11a is in communication with a water inlet pipe 31a of the three-way valve pipe 31 through the first water pump 33, a water return pipe 31b of the three-way valve pipe 31 is in communication with the fluid medium outlet 21b, and a water mixing pipe 31c of the three-way valve pipe 31 is in communication with the water outlet 11b. The device 100 further includes a pressure regulating assembly 34, and the pressure regulating assembly 34 includes a pressure sensor 34a and a pressure relief valve (not shown). The pressure sensor 34a is configured to collect a water pressure signal in the water pipe assembly 10, and the pressure relief valve is disposed at the water inlet 11a side and is configured to actively open when the water pressure in the water pipe assembly 10 exceeds a first set threshold, so as to discharge at least part of fluid in the water pipe assembly 10. The device 100 further includes a control system 80, and the control system 80 includes a main control module 81 and a power supply module 82. An output end of the power supply module 82 is respectively connected to a power supply end of the main control module 81, a power supply end of the pressure sensor 34a, and a power supply end of the first water pump 33. An output end of the pressure sensor 34a is connected to an input end of the main control module 81, and an output end of the main control module 81 is connected to an input end of the first water pump 33. The control system 80 is configured to: when the water pressure signal collected by the pressure sensor 34a exceeds a second set threshold, control the first water pump 33 to stop working; where the first set threshold is greater than the second set threshold.

According to the device 100 of the third embodiment of the present application, through cooperation of the water pipe assembly 10, the fluid medium pipe assembly 20, the valve body assembly 30, and the reservoir 50, the fluid medium can be stably extracted and mixed, realizing automatic addition of shower medium and enriching the shower function. In addition, the device 100 is provided with differentiated dual water pressure protection thresholds, with the first set threshold greater than the second set threshold, thereby forming a graded safety protection. When the water pressure in the water pipe assembly 10 exceeds the second set threshold, the control system 80 can quickly control the first water pump 33 to stop, avoiding damage to the device 100 due to water pressure overload. When the water pressure continues to rise and exceeds the first set threshold, the pressure relief valve at the water inlet 11a side automatically opens to release pressure, thereby quickly reducing the pipeline water pressure. The device 100 of the third embodiment combines electrical control shutdown protection with mechanical pressure relief protection, providing dual protection for pipeline and equipment safety, thereby effectively solving the problem of single water pressure protection and poor safety in traditional devices, and greatly improving the stability and safety of device operation.

In some specific examples of the present application, the control system 80 further includes a communication module (e.g. a radio frequency receiver) 83, and the main control module 81 is in bidirectional communication connection with the communication module 83. The control system 80 is further configured to: when the water pressure signal collected by the pressure sensor 34a exceeds the second set threshold, push, through the communication module 83, warning information indicating that the water pipe assembly 10 of the device is in water pressure abnormality to an external terminal device. The device 100 of the present embodiment may further push water pressure abnormality warning to the user through the communication module 83, thereby realizing intelligent fault reminding.

In some specific examples of the present application, the second set threshold is a fixed setting threshold when the device 100 leaves the factory; or, the second set threshold is a user-defined pressure threshold pushed by the external terminal device to the main control module 81, and the user-defined pressure threshold is less than or equal to the fixed setting threshold when the device 100 leaves the factory. In some embodiments, the second set threshold supports two setting modes: a factory fixed threshold may be taken to meet routine standardized protection requirements; or an external terminal may can push a user-defined pressure threshold to the main control module 81, and the user-defined threshold is not greater than the factory fixed threshold, adapting to personalized protection requirements of different usage scenarios, with flexible and adjustable protection modes.

In some specific examples of the present application, the pressure sensor 34a is configured to collect the water pressure signal of the water pipe assembly 10, including that: the pressure sensor 34a is disposed at the water inlet 11a side, and the pressure sensor 34a is configured to collect a water pressure signal at the water inlet 11a side; and/or, the pressure sensor 34a is disposed at the water outlet 11b side, and the pressure sensor 34a is configured to collect a water pressure signal at the water outlet 11b side.

In some specific examples of the present application, the pressure relief valve adopts a conventional structure known in the art, so the present embodiment does not show it in the drawings or describe it in detail. Specifically, the pressure relief valve may include a valve body, an opening/closing valve core, and a preloaded elastic member. The valve body is connected in parallel with the water inlet 11a side, the preloaded elastic member is configured to provide a preload pressure to the opening/closing valve core so that the opening/closing valve core seals an overflow passage of the valve body, and the water pipe assembly 10 can discharge at least part of fluid therein through the overflow passage. The pressure relief valve is disposed at the water inlet 11a side and is configured to actively open when the water pressure in the water pipe assembly 10 exceeds the first set threshold, including that: when the water pressure in the water pipe assembly 10 exceeds the first set threshold, the opening/closing valve core receives a water pressure thrust from the water inlet 11a side so that the opening/closing valve core overcomes the preload pressure provided by the preloaded elastic member, thereby causing the preloaded elastic member to displace, so as to make the opening/closing valve core disengage from the overflow passage of the valve body. In the present embodiment, the pressure relief valve is connected in parallel at the water inlet 11a side, enabling rapid response when the water pressure at the water inlet 11a side is overpressure.

In some specific examples of the present application, when the water pressure in the water pipe assembly 10 falls back below the first set threshold, the preloaded elastic member resets to provide the preload pressure, so as to cause the opening/closing valve core to reseal the overflow passage of the valve body. In the present embodiment, the pressure relief valve relies on the preloaded elastic member to automatically open for pressure relief when water pressure exceeds the threshold, and automatically close and reset when water pressure returns, without manual regulation.

In some specific examples of the present application, the water pipe assembly 10 further includes a bypass pipe 12 and a flow regulator 13. A water inlet end 12a of the bypass pipe 12 is in communication with the water inlet 11a and the water inlet pipe 31a respectively, a water outlet end 12b of the bypass pipe 12 is in communication with the water outlet 11b and the water mixing pipe 31c respectively, and the flow regulator 13 is disposed between the water inlet end 12a of the bypass pipe 12 and the water outlet end 12b of the bypass pipe 12. In the present embodiment, the technical advantages brought by the bypass pipe 12 and the flow regulator 13 to the device 100 may refer to the second embodiment, and will not be repeated here.

It can be understood that the remaining structures of the third embodiment may follow the corresponding structures of the first and second embodiments, and the structures of the present embodiment may be combined with the corresponding structures of the fourth and fifth embodiments described later, adapting to different configuration requirements.

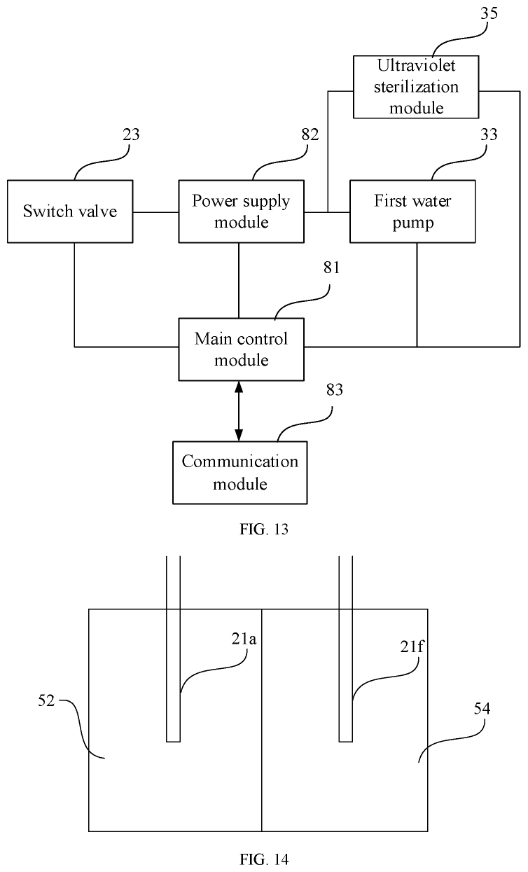

A device 100 for adding a fluid medium to a shower system according to a fourth embodiment of the present application will be described below with reference to FIGS. 13-14 and FIGS. 1-11.

The device 100 for adding a fluid medium to a shower system according to the fourth embodiment of the present application includes: a water pipe assembly 10. The water pipe assembly 10 includes a water pipe 11, the water pipe 11 has a water inlet 11a and a water outlet 11b, where the water inlet 11a is configured to be connected with a water source connector of the shower system for introducing water, and the water outlet 11b is configured to be connected with a showerhead of the shower system. The device 100 further includes a reservoir 50 for storing the fluid medium, and the reservoir 50 includes a storage chamber (for example, a first chamber 52 in FIG. 14) and a cleaning chamber 54 which are independently arranged. The storage chamber is configured to accommodate a first fluid medium for body washing of a user, and the cleaning chamber 54 is configured to accommodate a cleaning agent for cleaning pipelines of the device 100. The device 100 further includes a fluid medium pipe assembly 20, and the fluid medium pipe assembly 20 includes a suction pipe 21 and a switch valve 23. The suction pipe 21 has a fluid medium inlet 21a and a fluid medium outlet 21b, and the fluid medium outlet 21b is connected with the water pipe assembly 10. The suction pipe 21 includes a communication portion 21e, a care medium suction portion (such as first suction portion 21c), and a cleaning agent suction portion 21f. A first end of the care medium suction portion extends into the storage chamber, and a second end of the care medium suction portion is connected with a liquid inlet end of the communication portion 21e. A first end of the cleaning agent suction portion 21f extends into the cleaning chamber 54, and a second end of the cleaning agent suction portion 21f is connected with the liquid inlet end of the communication portion 21e. The first end of the care medium suction portion and the first end of the cleaning agent suction portion 21f respectively constitute the fluid medium inlet 21a, a liquid outlet end of the communication portion 21e constitutes the fluid medium outlet 21b, and the switch valve 23 is configured to allow the communication portion 21e to be selectively in communication with the care medium suction portion or the cleaning agent suction portion 21f. The device 100 further includes a valve body assembly 30, and the valve body assembly 30 includes a first water pump 33 and a three-way valve pipe 31. The water inlet 11a is in communication with a water inlet pipe 31a of the three-way valve pipe 31 through the first water pump 33, a water return pipe 31b of the three-way valve pipe 31 is in communication with the fluid medium outlet 21b, and a water mixing pipe 31c of the three-way valve pipe 31 is in communication with the water outlet 11b. The device 100 further includes a control system 80, and the control system 80 includes a main control module (e.g. a controller) 81 and a power supply module (e.g. a battery) 82. An output end of the power supply module 82 is respectively connected to a power supply end of the main control module 81, a power supply end of the switch valve 23, and a power supply end of the first water pump 33, and an output end of the main control module 81 is connected to an input end of the switch valve 23. The control system 80 is configured to: upon receiving a first signal indicating that the user has finished showering or a second signal indicating that a standing time after the user finishes showering reaches a first preset delay threshold, control the switch valve 23 to allow the communication portion 21e to be in communication with the cleaning agent suction portion 21f, and control the first water pump 33 to work so that the cleaning agent in the cleaning chamber 54 flows into the water pipe assembly 10 to clean at least part of pipelines of the water pipe assembly 10.

According to the device 100 of the fourth embodiment of the present application, the reservoir 50 includes a storage chamber and a cleaning chamber 54, cooperating with the care medium suction portion, the cleaning agent suction portion 21f, and the switch valve 23 of the suction pipe 21, so that the communication portion 21e selectively communicates with the care medium suction portion or the cleaning agent suction portion 21f. The control system 80, upon receiving the first signal indicating that the user has finished showering or the second signal indicating that the standing time after finishing showering reaches the first preset delay threshold, controls the switch valve 23 to allow the communication portion 21e to be in communication with the cleaning agent suction portion 21f, and controls the first water pump 33 to work, extracting the cleaning agent in the cleaning chamber 54 into the water pipe assembly 10 to clean at least part of pipelines of the water pipe assembly 10. This device may effectively flush away care medium residues adhered to the inner wall of the pipelines, prevent long-term accumulation and scaling of care products, avoid bacterial and mold growth inside the pipelines of the device 100, and ensure the cleanliness of the fluid delivered by the device 100. In addition, the self-cleaning process does not require manual disassembly and cleaning of the pipelines, which is convenient to maintain and may improve the working stability and hygiene safety of the device 100.

In some embodiments, the storage chamber has only one chamber, for example, the first chamber 52 shown in FIG. 14. In other embodiments, the storage chamber includes a plurality of chambers. It can be understood that, for example, as shown in FIG. 4, the storage chamber includes a first chamber 52 and a second chamber 53 which are independent from each other, where the first chamber 52 is configured to accommodate a first fluid medium, and the second chamber 53 is configured to accommodate a second fluid medium different from the first fluid medium. The care medium suction portion includes a first suction portion 21c and a second suction portion 21d. The communication method and control method between the first chamber 52, the second chamber 53, and the fluid medium outlet 21b may refer to the design of the first embodiment, and will not be repeated here.

In some specific examples of the present application, the device 100 further includes a reversing valve assembly 90 capable of controlling flow direction, and the reversing valve assembly 90 is configured to establish a forward flow path from the fluid medium inlet 21a to the fluid medium outlet 21b and a reverse flow path from the fluid medium outlet 21b to the fluid medium inlet 21a. An output end of the power supply module 82 is connected to a power supply end of the reversing valve assembly 90, and an output end of the main control module 81 is connected to an input end of the reversing valve assembly 90. The controlling the first water pump 33 to work so that the cleaning agent in the cleaning chamber 54 flows into the water pipe assembly 10 to clean at least part of pipelines of the water pipe assembly 10 may include: controlling the reversing valve assembly 90 to establish the forward flow path, and controlling the first water pump 33 to work so that the cleaning agent in the cleaning chamber 54 flows through the forward flow path into the water pipe assembly 10, for causing the cleaning agent to perform forward cleaning of the device 100. In the present embodiment, the forward flow path established by the reversing valve assembly 90 enables the cleaning agent in the cleaning chamber to be stably delivered along the predetermined direction from the fluid medium inlet 21a to the fluid medium outlet 21b. With the cooperation of the main control module 81, the power supply module 82, and the first water pump 33, the forward cleaning operation of the water pipe assembly 10 can be automatically completed.

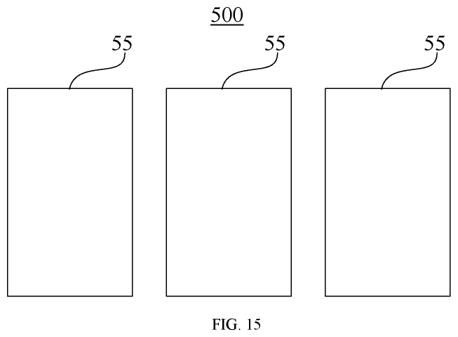

It can be understood that the reservoir 50 includes a storage chamber and a cleaning chamber 54 which are independently arranged. This design may refer to the design of the consumable cartridge 55 in FIG. 15, and its structure and assembly method with the device 100 may refer to the description of the fifth embodiment, which will not be repeated here.

In some specific examples of the present application, the control system 80 is further configured to: after the cleaning agent performs forward cleaning of at least part of pipelines of the water pipe assembly 10, control the switch valve 23 to allow the communication portion 21e to be in communication with the care medium suction portion, control the reversing valve assembly 90 to establish the reverse flow path, and control the first water pump 33 to work so that the cleaning agent remaining in said at least part of pipelines flows through the reverse flow path into the storage chamber, for causing the cleaning agent to perform reverse cleaning of the device 100. In the present embodiment, the reversing valve assembly 90 can respectively establish the forward flow path and the reverse flow path, cooperating with the switch valve 23, the first water pump 33, and the control system 80 to realize bidirectional cleaning operation. After the forward cleaning is completed, the main control module 81 may control the switch valve 23 to switch the communication portion 21e to connect with the care medium suction portion, and control the reversing valve assembly 90 to establish the reverse flow path from the fluid medium outlet 21b to the fluid medium inlet 21a, so that the cleaning agent remaining in the pipelines flows back reversely. The present embodiment, through alternating forward and reverse flushing, may effectively peel off stubborn medium residues that are difficult to remove by forward cleaning, thereby further improving the self-cleaning effect and pipeline cleanliness of the device 100.

In some specific examples of the present application, the device 100 further includes an ultraviolet sterilization module 35, and the ultraviolet sterilization module 35 is disposed in the reservoir 50 or in pipelines of the fluid medium pipe assembly 20. An output end of the power supply module 82 is connected to a power supply end of the ultraviolet sterilization module, and an output end of the main control module 81 is connected to an input end of the ultraviolet sterilization module. The control system 80 is further configured to: upon receiving a third signal indicating that a standing time after the user finishes showering reaches a second preset delay threshold, control the ultraviolet sterilization module to turn on so as to inhibit generation of bacteria in the reservoir 50 or in the pipelines of the fluid medium pipe assembly 20.

In some specific examples of the present application, the control system 80 further includes a communication module 83, and the main control module 81 is in bidirectional communication connection with the communication module 83. The control system 80 is further configured to: after controlling the first water pump 33 to work so that the cleaning agent in the cleaning chamber 54 cleans at least part of pipelines of the water pipe assembly 10, push, through the communication module 83, prompt information indicating that the device 100 has completed self-cleaning to an external terminal device. In the present embodiment, the control system 80 is additionally provided with a communication module 83, enabling the main control module 81 to actively push corresponding completion prompt information to the external terminal device after the device 100 completes the pipeline self-cleaning operation, allowing the user to timely know the cleaning working state of the device, thereby realizing perceivable and traceable device operation status and improving the intelligence level and user experience of the device.

In some specific examples of the present application, the first preset delay threshold is a user-defined preset delay threshold pushed by the external terminal device to the main control module 81, and the user-defined preset delay threshold is greater than or equal to the fixed preset delay threshold when the device 100 leaves the factory. In the present embodiment, the first preset delay threshold may adopt a factory fixed parameter of the device 100, or an external terminal device may push a user-defined preset delay threshold to the main control module 81. The user-defined preset delay threshold is not less than the factory fixed preset delay threshold, which may flexibly adapt the self-cleaning start timing according to user habits and usage scenarios, taking into account both device versatility and scenario adaptability.

In some specific examples of the present application, the main control module 81 is configured with a local non-volatile storage unit, and the local non-volatile storage unit is configured to store, item by item, a completion record corresponding to each self-cleaning. This design may locally save historical self-cleaning records, avoiding loss due to power failure or restart, and facilitating user review of device cleaning operation status. Further, in cooperation with the information push function of the communication module 83, it is convenient for the user to grasp the cleaning condition of the device.

In some specific examples of the present application, the water pipe assembly 10 further includes a bypass pipe 12 and a flow regulator 13. A water inlet end 12a of the bypass pipe 12 is in communication with the water inlet 11a and the water inlet pipe 31a respectively, a water outlet end 12b of the bypass pipe 12 is in communication with the water outlet 11b and the water mixing pipe 31c respectively, and the flow regulator 13 is disposed between the water inlet end 12a of the bypass pipe 12 and the water outlet end 12b of the bypass pipe 12. In the present embodiment, the technical advantages brought by the bypass pipe 12 and the flow regulator 13 to the device 100 may refer to the second embodiment, and will not be repeated here.

A device 100 for adding a fluid medium to a shower system according to a fifth embodiment of the present application will be described below with reference to FIG. 15 and FIGS. 1-11.

The device 100 for adding a fluid medium to a shower system according to the fifth embodiment of the present application includes a box body 40 and a liquid storage assembly 500, where the box body 40 and the liquid storage assembly 500 are detachably connected. The box body 40 is internally provided with a water pipe assembly 10, and the water pipe assembly 10 includes a water pipe 11. The water pipe 11 has a water inlet 11a and a water outlet 11b, the water inlet 11a is configured to be connected with a water source connector of the shower system for introducing water, and the water outlet 11b is configured to be connected with a showerhead of the shower system. The box body 40 is further internally provided with a fluid medium pipe assembly 20, and the fluid medium pipe assembly 20 includes a suction pipe 21 and a switch valve 23. The suction pipe 21 has a fluid medium outlet 21b and a plurality of mutually independent fluid medium inlets 21a. The fluid medium outlet 21b is connected with the water pipe assembly 10, a communication portion 21e is provided inside the suction pipe 21, the plurality of fluid medium inlets 21a are respectively in communication with the fluid medium outlet 21b via the communication portion 21e, and the switch valve 23 is configured to switch a flow path of the communication portion 21e so that the communication portion 21e selectively connects any one or more of the fluid medium inlets 21a with the fluid medium outlet 21b. The box body 40 is further internally provided with a valve body assembly 30, and the valve body assembly 30 includes a first water pump 33 and a three-way valve pipe 31. The water inlet 11a is in communication with a water inlet pipe 31a of the three-way valve pipe 31 through the first water pump 33, a water return pipe 31b of the three-way valve pipe 31 is in communication with the fluid medium outlet 21b, and a water mixing pipe 31c of the three-way valve pipe 31 is in communication with the water outlet 11b. The liquid storage assembly 500 includes a plurality of mutually independent consumable cartridges 55, each consumable cartridge 55 having a chamber (such as a first chamber 52) for placing a fluid medium. The box body 40 and the liquid storage assembly 500 are detachably connected, including that: the box body 40 is provided with a plurality of connection positions for detachably connecting with the consumable cartridges 55, and the plurality of mutually independent consumable cartridges 55 can be detachably connected to the connection positions in a one-to-one correspondence, so that each fluid medium inlet 21a of the suction pipe 21 extends into the chamber of a corresponding consumable cartridge 55.

According to the device 100 of the fifth embodiment of the present application, a modular structure design with detachable connection between the box body 40 and the liquid storage assembly 500 is adopted. The liquid storage assembly 500 is provided with a plurality of mutually independent consumable cartridges 55, cooperating with the corresponding plurality of connection positions on the box body 40, to realize independent assembly, disassembly, and replacement of the consumable cartridges 55. This modular liquid storage system abandons the integrated fixed liquid storage structure. Each consumable cartridge 55 can be independently detached, replaced, and refilled without the need to disassemble the whole device, thereby greatly simplifying the operation of adding, replacing and maintaining the fluid medium, and providing higher convenience. In addition, the chambers of the plurality of mutually independent consumable cartridges 55 may respectively contain different types of fluid media, capable of accommodating various care media such as shampoo, conditioner, shower gel, fragrance, essence, scalp care liquid, body skin care preparation, etc. With the switch valve 23 of the fluid medium pipe assembly 20 switching the flow path, on-demand selection, and delivery of different fluid media may be realized, greatly enriching the medium adding function of the shower system and adapting to diverse care usage scenarios. Moreover, the independently separated consumable cartridges 55 may effectively prevent different fluid media from mixing and cross-contaminating each other, ensuring the purity and effectiveness of various care media, and significantly improving the overall versatility, expandability and practicability of the structure.

It can be understood that the box body 40 is provided with a plurality of connection positions for detachably connecting the consumable cartridges 55, and the plurality of independent consumable cartridges 55 are assembled to the connection positions in a one-to-one correspondence, so that each fluid medium inlet 21a of the suction pipe 21 extends into the chamber of a corresponding consumable cartridge 55. The detachable connection between the consumable cartridge 55 and the box body 40 may adopt various structural forms, specifically as follows. In one specific embodiment, the connection position defines a plug-in slot, and an outer wall of the consumable cartridge 55 is provided with a matching plug-in rib, where the plug-in slot and the plug-in rib can be plugged and fitted to realize plug-in detachable connection. This structure is convenient for assembly and disassembly, stable in positioning after assembly, and may effectively prevent the consumable cartridge 55 from shifting or falling off, ensuring that the fluid medium inlet 21a stably extends into the chamber of the consumable cartridge 55. In another specific embodiment, the connection position defines a threaded connection hole, and an end of the consumable cartridge 55 is provided with a matching external thread, where threaded detachable connection can be realized through screw connection. This connection method has good positioning and sealing effects, firm connection, may effectively prevent the consumable cartridge 55 from loosening and shifting during device operation, and is suitable for long-term use scenarios, convenient for medium supply and consumable replacement. In yet another specific embodiment, the connection position is provided with a limiting card seat with an elastic buckle, and an outer wall of the consumable cartridge 55 defines a limiting groove, where snap-fit detachable connection can be realized through the engagement of the elastic buckle and the limiting groove. This structure can be assembled and disassembled without tools, is simple to operate, reliable in limiting and fixing, and is suitable for use scenarios with frequent assembly and disassembly for maintenance. In still another specific embodiment, the connection position and a butt end of the consumable cartridge 55 are provided with mutually matching magnetic members, and through magnetic attraction cooperation, magnetic detachable connection can be realized. This structure has no mechanical wear during assembly and disassembly, accurate alignment, efficient operation, and further simplifies the device maintenance process.

The above various detachable connection structures can all realize convenient assembly and disassembly and precise positioning of the consumable cartridges 55, ensuring that the fluid medium inlet 21a stably extends into the chamber of the corresponding consumable cartridge 55, and can adapt to different assembly and maintenance scenarios, with excellent structural adaptability and practicability.

In some specific examples of the present application, the device 100 further includes a control system 80, and the control system 80 includes a main control module 81 and a power supply module 82. An output end of the power supply module 82 is respectively connected to a power supply end of the main control module 81, a power supply end of the switch valve 23, and a power supply end of the first water pump 33, and an output end of the main control module 81 is connected to an input end of the first water pump 33 and an input end of the switch valve 23. The control system 80 is configured to: control the switch valve 23 to allow the communication portion 21e to connect any one or more of the fluid medium inlets 21a with the fluid medium outlet 21b, and control the first water pump 33 to work so that the fluid medium in the consumable cartridge 55 flows into the water pipe assembly 10, for causing the fluid medium to mix with water in the water pipe assembly 10 and then flow out from the water outlet 11b. In the present embodiment, the device 100 is provided with a control system 80, which can supply power uniformly to the main control module 81, the switch valve 23, and the first water pump 33 through the power supply module 82, thereby ensuring stable and coordinated operation of each electrical component. In addition, through unified output of control signals by the main control module 81, regulation of the switch valve 23 and the first water pump 33 may be realized. The control system 80 may control the switch valve 23 through the main control module 81 to allow the communication portion 21e to connect the corresponding fluid medium inlet 21a with the fluid medium outlet 21b as needed, and cooperate with the first water pump 33 to stably deliver the fluid medium in the consumable cartridge 55 into the water pipe assembly 10, so that the fluid medium may fully mix with the water in the pipeline and then be output from the water outlet 11b.

In some specific examples of the present application, the control system 80 further includes a communication module 83, and the main control module 81 is in bidirectional communication connection with the communication module 83. The control system 80 is further configured to: receive a user-defined shower instruction sent by an external terminal device, where the user-defined shower instruction includes a connection timing sequence set by the user for the switch valve 23 to connect each chamber of the consumable cartridges 55 with the fluid medium outlet 21b. The controlling the switch valve 23 to allow the communication portion 21e to connect any one or more of the fluid medium inlets 21a with the fluid medium outlet 21b may include: according to the connection timing sequence in the user-defined shower instruction, controlling the switch valve 23 to connect the chamber of the corresponding consumable cartridge 55 with the fluid medium outlet 21b according to the connection timing sequence. In the operation of the device 100 of the present embodiment, the main control module 81 may precisely control the switch valve 23 according to the user-defined connection timing sequence, connecting the chamber of the corresponding consumable cartridge 55 with the fluid medium outlet 21b as needed, thereby realizing timed and personalized delivery of different fluid media.

In some specific examples of the present application, the device 100 further includes a reversing valve assembly 90 capable of controlling flow direction, and the reversing valve assembly 90 is disposed between the fluid medium inlet 21a and the fluid medium outlet 21b. The reversing valve assembly 90 is capable of controlling water to flow from the fluid medium outlet 21b to the fluid medium inlet 21a, and the reversing valve assembly 90 is further capable of controlling the fluid medium to flow from the fluid medium inlet 21a to the fluid medium outlet 21b. The structure and control method of the reversing valve assembly 90 may refer to the design of the first embodiment, and will not be repeated here.

In some specific examples of the present application, the water pipe assembly 10 further includes a bypass pipe 12 and a flow regulator 13. A water inlet end 12a of the bypass pipe 12 is in communication with the water inlet 11a and the water inlet pipe 31a respectively, a water outlet end 12b of the bypass pipe 12 is in communication with the water outlet 11b and the water mixing pipe 31c respectively, and the flow regulator 13 is disposed between the water inlet end 12a of the bypass pipe 12 and the water outlet end 12b of the bypass pipe 12. In the present embodiment, the technical advantages brought by the bypass pipe 12 and the flow regulator 13 to the device 100 may refer to the second embodiment, and will not be repeated here.

A shower system according to an embodiment of the present application, includes a showerhead (not shown) and a water source connector for introducing water. A water inter (not shown) of the showerhead is in communication with a water outlet (not shown) of the water source connector. The shower system further includes the device 100 for adding a fluid medium to the shower system as described above. The water outlet of the water source connector is further in communication with the water inlet 11a, and the water inter of the showerhead is further in communication with the water outlet 11b.

The shower system according to the embodiment of the present application enables the user to control the supply of the fluid medium easily while showering. In addition, the shower system according to the embodiment of the present application has other advantages as described for the above device 100 for adding a fluid medium to a shower system, which will not be repeated here.

In some specific examples of the present application, the system further includes a four-way valve pipe (not shown) provided with a first water inlet, a first water outlet, a second water inlet and a second water outlet. The first water inlet is in communication with the water outlet of the water source connector, the first water outlet is in communication with the water inlet 11a, the second water inlet is in communication with the water outlet 11b, and the second water outlet is in communication with the water inlet of the showerhead. When the user does not need the fluid medium, water reaches the user in two branches in which the first branch of water flows through the first water inlet and the second water outlet, and the second branch of water flows through the first water inlet, the first water outlet, the second water inlet and the second water outlet. As such, the supply of the fluid medium is stopped through controlling the venturi switch 22 by the user. When the user needs the fluid medium, water reaches the user through the first water inlet and the second water outlet, and the mixture of the liquid medium and water reaches the user through the second water inlet and the second water outlet.

In the description of the present application, it is should be understood that terms “first”, “second”, and “third” are only used for the purpose of description, but are not intended to indicate or imply the relative importance or implicitly indicate the number of technical features indicated. Therefore, a feature defined by “first”, “second” and “third” may explicitly or implicitly includes one or more such features. In the description of the present application, the term “a plurality of” indicates two and more, unless expressly specified otherwise.

In the description of the present application, reference to “an embodiment”, “some embodiments”, “an example”, “specific examples” or “some examples” herein means that the specific feature, structure, or characteristic described in conjunction with the embodiments or examples may be included in at least one embodiment or example of the present application. In this description, the schematic representations of the above terms do not necessarily refer to the same embodiment or example. Moreover, the described specific features, structures, materials or characteristics can be combined in any one or more embodiments or examples in a suitable manner. In addition, those skilled in the art may combine the different embodiments or examples and the features of the different embodiments or examples described in this specification when they are not mutually contradict.

Although the embodiments of the present application have been shown and described, those of ordinary skill in the art may understand that various changes, modifications, substitutions and variations may be made to these embodiments without departing from the principle and spirit of the present application. The scope of the present application is defined by the claims and their equivalents.

What is claimed is:

1. A device for adding a fluid medium to a shower system, comprising:
a water pipe assembly, comprising a water pipe; wherein the water pipe has a water inlet and a water outlet, the water inlet is configured to be connected with a water source connector of the shower system for introducing water, and the water outlet is configured to be connected with a showerhead of the shower system;
a fluid medium pipe assembly, comprising a suction pipe; wherein the suction pipe has a fluid medium inlet and a fluid medium outlet, and the fluid medium outlet is connected with the water pipe assembly;
a reservoir for storing the fluid medium; wherein the suction pipe extends into the reservoir;
a valve body assembly, comprising a first water pump and a three-way valve pipe; wherein the water inlet is in communication with a water inlet pipe of the three-way valve pipe through the first water pump, a water return pipe of the three-way valve pipe is in communication with the fluid medium outlet, and a water mixing pipe of the three-way valve pipe is in communication with the water outlet;
a pressure regulating assembly, comprising a pressure sensor and a pressure relief valve; wherein the pressure sensor is configured to collect a water pressure signal of the water pipe assembly, and the pressure relief valve is disposed at a water inlet side and is configured to actively open in a case where the water pressure signal in the water pipe assembly exceeds a first set threshold, for discharging at least part of fluid in the water pipe assembly; and
a control system, comprising a main control module and a power supply module; wherein an output end of the power supply module is respectively connected to a power supply end of the main control module, a power supply end of the pressure sensor, and a power supply end of the first water pump; an output end of the pressure sensor is connected to an input end of the main control module, and an output end of the main control module is connected to an input end of the first water pump;
wherein the control system is configured to: in a case where the water pressure signal collected by the pressure sensor exceeds a second set threshold, control the first water pump to stop working; wherein the first set threshold is greater than the second set threshold.

2. The device for adding a fluid medium to a shower system according to claim 1, wherein the control system further comprises a communication module, and the main control module is in bidirectional communication connection with the communication module;
the control system is further configured to: in a case where the water pressure signal collected by the pressure sensor exceeds the second set threshold, push, through the communication module, warning information indicating that the water pipe assembly of the device is in water pressure abnormality to an external terminal device.

3. The device for adding a fluid medium to a shower system according to claim 2, wherein the second set threshold is a fixed setting threshold when the device leaves a factory; or,
the second set threshold is a user-defined pressure threshold pushed by the external terminal device to the main control module, and the user-defined pressure threshold is less than or equal to the fixed setting threshold when the device leaves the factory.

4. The device for adding a fluid medium to a shower system according to claim 1, wherein the pressure sensor is disposed at the water inlet side, and the pressure sensor is configured to collect the water pressure signal at the water inlet side; and/or, the pressure sensor is disposed at a water outlet side, and the pressure sensor is configured to collect the water pressure signal at the water outlet side.

5. The device for adding a fluid medium to a shower system according to claim 1, wherein the pressure relief valve comprises a valve body, an opening/closing valve core, and a preloaded elastic member; the valve body is connected in parallel with the water inlet side; the preloaded elastic member is configured to provide a preload pressure to the opening/closing valve core, for causing the opening/closing valve core to seal an overflow passage of the valve body; the water pipe assembly is capable of discharging at least part of the fluid in the water pipe assembly through the overflow passage;
wherein, in a case where the water pressure signal in the water pipe assembly exceeds the first set threshold, the opening/closing valve core receives a water pressure thrust from the water inlet side, for causing the opening/closing valve core to overcome the preload pressure provided by the preloaded elastic member, and thereby causing the preloaded elastic member to displace to make the opening/closing valve core disengage from the overflow passage of the valve body.

6. The device for adding a fluid medium to a shower system according to claim 5, wherein in a case where the water pressure signal in the water pipe assembly falls back below the first set threshold, the preloaded elastic member resets to provide the preload pressure, for causing the opening/closing valve core to reseal the overflow passage of the valve body.

7. The device for adding a fluid medium to a shower system according to claim 1, wherein the water pipe assembly further comprises a bypass pipe and a flow regulator; a water inlet end of the bypass pipe is in communication with the water inlet and the water inlet pipe, and a water outlet end of the bypass pipe is in communication with the water outlet and the water mixing pipe; wherein the flow regulator is provided between the water inlet end of the bypass pipe and the water outlet end of the bypass pipe.

8. A device for adding a fluid medium to a shower system, comprising:
a water pipe assembly, comprising a water pipe; wherein the water pipe has a water inlet and a water outlet, the water inlet is configured to be connected with a water source connector of the shower system for introducing water, and the water outlet is configured to be connected with a showerhead of the shower system;
a reservoir for storing the fluid medium; wherein the reservoir comprises a storage chamber and a cleaning chamber that are independently arranged; the storage chamber is configured to accommodate a care medium for body washing of a user, and the cleaning chamber is configured to accommodate a cleaning agent for cleaning pipelines of the device;
a fluid medium pipe assembly, comprising a suction pipe and a switch valve; wherein the suction pipe has a fluid medium inlet and a fluid medium outlet, and the fluid medium outlet is connected with the water pipe assembly; wherein the suction pipe comprises a communication portion, a care medium suction portion, and a cleaning agent suction portion; a first end of the care medium suction portion extends into the storage chamber, and a second end of the care medium suction portion is connected with a liquid inlet end of the communication portion; a first end of the cleaning agent suction portion extends into the cleaning chamber, and a second end of the cleaning agent suction portion is connected with the liquid inlet end of the communication portion; the first end of the care medium suction portion and the first end of the cleaning agent suction portion respectively constitute the fluid medium inlet, and a liquid outlet end of the communication portion constitutes the fluid medium outlet; the switch valve is configured to allow the communication portion to be selectively in communication with the care medium suction portion or the cleaning agent suction portion;
a valve body assembly, comprising a first water pump and a three-way valve pipe; wherein the water inlet is in communication with a water inlet pipe of the three-way valve pipe through the first water pump, a water return pipe of the three-way valve pipe is in communication with the fluid medium outlet, and a water mixing pipe of the three-way valve pipe is in communication with the water outlet; and
a control system, comprising a main control module and a power supply module; wherein an output end of the power supply module is respectively connected to a power supply end of the main control module, a power supply end of the switch valve, and a power supply end of the first water pump, and an output end of the main control module is connected to an input end of the switch valve;
wherein the control system is configured to: upon receiving a first signal indicating that the user has finished showering or a second signal indicating that a standing time after the user finishes showering reaches a first preset delay threshold, control the switch valve to allow the communication portion to be in communication with the cleaning agent suction portion, and control the first water pump to work, for causing the cleaning agent in the cleaning chamber to flow into the water pipe assembly to clean at least part of pipelines of the water pipe assembly.

9. The device for adding a fluid medium to a shower system according to claim 8, wherein the device further comprises a reversing valve assembly capable of controlling flow direction; the reversing valve assembly is configured to establish a forward flow path from the fluid medium inlet to the fluid medium outlet and configured to establish a reverse flow path from the fluid medium outlet to the fluid medium inlet; an output end of the power supply module is connected to a power supply end of the reversing valve assembly, and an output end of the main control module is connected to an input end of the reversing valve assembly;
the controlling the first water pump to work, for causing the cleaning agent in the cleaning chamber to flow into the water pipe assembly to clean at least part of pipelines of the water pipe assembly comprises: controlling the reversing valve assembly to establish the forward flow path, and controlling the first water pump to work, for causing the cleaning agent in the cleaning chamber to flow through the forward flow path into the water pipe assembly, and thereby causing the cleaning agent to perform forward cleaning of the device.

10. The device for adding a fluid medium to a shower system according to claim 9, wherein the control system is further configured to: after the cleaning agent performs the forward cleaning of the at least part of the pipelines of the water pipe assembly, control the switch valve to allow the communication portion to be in communication with the care medium suction portion, control the reversing valve assembly to establish the reverse flow path, and control the first water pump to work, for causing the cleaning agent remaining in the at least part of the pipelines to flow through the reverse flow path into the storage chamber, and thereby causing the cleaning agent to perform reverse cleaning of the device.

11. The device for adding a fluid medium to a shower system according to claim 8, wherein the device further comprises an ultraviolet sterilization module; the ultraviolet sterilization module is disposed in the reservoir or in pipelines of the fluid medium pipe assembly; an output end of the power supply module is connected to a power supply end of the ultraviolet sterilization module, and an output end of the main control module is connected to an input end of the ultraviolet sterilization module;
the control system is further configured to: upon receiving a third signal indicating that a standing time after the user finishes showering reaches a second preset delay threshold, control the ultraviolet sterilization module to turn on, for inhibiting generation of bacteria in the reservoir or in the pipelines of the fluid medium pipe assembly.

12. The device for adding a fluid medium to a shower system according to claim 8, wherein the control system further comprises a communication module, and the main control module is in bidirectional communication connection with the communication module;
wherein the control system is further configured to: after controlling the first water pump to work for causing the cleaning agent in the cleaning chamber to clean the at least part of the pipelines of the water pipe assembly, push, through the communication module, prompt information indicating that the device has completed self-cleaning to an external terminal device; and/or,
wherein the first preset delay threshold is a user-defined preset delay threshold pushed by the external terminal device to the main control module, and the user-defined preset delay threshold is greater than or equal to a fixed preset delay threshold when the device leaves a factory.

13. The device for adding a fluid medium to a shower system according to claim 11, wherein the main control module is configured with a local non-volatile storage unit, and the local non-volatile storage unit is configured to store, item by item, a completion record corresponding to each self-cleaning; and/or,
wherein the water pipe assembly further comprises a bypass pipe and a flow regulator; a water inlet end of the bypass pipe is in communication with the water inlet and the water inlet pipe respectively; a water outlet end of the bypass pipe is in communication with the water outlet and the water mixing pipe respectively; the flow regulator is disposed between the water inlet end of the bypass pipe and the water outlet end of the bypass pipe.

14. A device for adding a fluid medium to a shower system, comprising: a box body and a liquid storage assembly; wherein the box body and the liquid storage assembly are detachably connected, and the box body is provided with:
a water pipe assembly, comprising a water pipe; wherein the water pipe has a water inlet and a water outlet; the water inlet is configured to be connected with a water source connector of the shower system for introducing water, and the water outlet is configured to be connected with a showerhead of the shower system;
a fluid medium pipe assembly, comprising a suction pipe and a switch valve; wherein the suction pipe has a fluid medium outlet and a plurality of fluid medium inlets, and the plurality of fluid medium inlets are mutually independent; the fluid medium outlet is connected with the water pipe assembly, a communication portion is provided inside the suction pipe, and the plurality of fluid medium inlets are respectively in communication with the fluid medium outlet via the communication portion; the switch valve is configured to switch a flow path of the communication portion, for causing the communication portion to selectively connect any one or more of the plurality of fluid medium inlets with the fluid medium outlet; and
a valve body assembly, comprising a first water pump and a three-way valve pipe; wherein the water inlet is in communication with a water inlet pipe of the three-way valve pipe through the first water pump, a water return pipe of the three-way valve pipe is in communication with the fluid medium outlet, and a water mixing pipe of the three-way valve pipe is in communication with the water outlet;
wherein the liquid storage assembly comprises a plurality of consumable cartridges that are mutually independent, and each consumable cartridge has a chamber for placing a fluid medium;
wherein the box body is provided with a plurality of connection positions for detachably connecting with the plurality of consumable cartridges, and the plurality of consumable cartridges are capable of being detachably connected to the plurality of connection positions in a one-to-one correspondence, enabling each fluid medium inlet of the suction pipe extends into the chamber of a corresponding consumable cartridge.

15. The device for adding a fluid medium to a shower system according to claim 14, wherein the device further comprises a control system, and the control system comprises a main control module and a power supply module; an output end of the power supply module is respectively connected to a power supply end of the main control module, a power supply end of the switch valve, and a power supply end of the first water pump, and an output end of the main control module is connected to an input end of the first water pump and an input end of the switch valve;
the control system is configured to: control the switch valve to allow the communication portion to connect any one or more of the plurality of fluid medium inlets with the fluid medium outlet, and control the first water pump to work, for causing the fluid medium in corresponding one or more of the plurality of consumable cartridge flows into the water pipe assembly, and thereby causing the fluid medium to mix with water in the water pipe assembly and then flow out from the water outlet.

16. The device for adding a fluid medium to a shower system according to claim 15, wherein the control system further comprises a communication module, and the main control module is in bidirectional communication connection with the communication module;
the control system is further configured to: receive a user-defined shower instruction sent by an external terminal device; wherein the user-defined shower instruction comprises a connection timing sequence set by a user for the switch valve to connect the chamber of each consumable cartridges with the fluid medium outlet;
wherein the controlling the switch valve to allow the communication portion to connect any one or more of the fluid medium inlets with the fluid medium outlet comprises: according to the connection timing sequence in the user-defined shower instruction, controlling the switch valve to connect the chamber of a corresponding consumable cartridge with the fluid medium outlet according to the connection timing sequence.

17. The device for adding a fluid medium to a shower system according to claim 16, wherein the device further comprises a reversing valve assembly configured to control a flow direction, the reversing valve assembly is disposed between the fluid medium inlet and the fluid medium outlet, wherein the reversing valve assembly is configured to control water to flow from the fluid medium outlet to the fluid medium inlet, and the reversing valve assembly is further configured to control the fluid medium to flow from the fluid medium inlet to the fluid medium outlet.

18. The device for adding a fluid medium to a shower system according to claim 14, wherein the water pipe assembly further comprises a bypass pipe and a flow regulator; a water inlet end of the bypass pipe is in communication with the water inlet and the water inlet pipe respectively, a water outlet end of the bypass pipe is in communication with the water outlet and the water mixing pipe respectively, and the flow regulator is disposed between the water inlet end of the bypass pipe and the water outlet end of the bypass pipe.

19. A shower system, comprising a showerhead and a water source connector for introducing water;
wherein a water inlet of the showerhead is in communication with a water outlet of the water source connector; the shower system further comprises the device for adding a fluid medium to the shower system according to claim 1.

20. The shower system according to claim 19, further comprising a four-way valve pipe provided with a first water inlet, a first water outlet, a second water inlet and a second water outlet; wherein the first water inlet is in communication with the water outlet of the water source connector, the first water outlet is in communication with the water inlet of the water pipe, the second water inlet is in communication with the water outlet of the water pipe, and the second water outlet is in communication with the water inlet of the showerhead.
